(19) United States
(12) Patent Application Publication
(10) Pub. No.: US 2009/0280484 A1
(43) Pub. Date: Nov. 12, 2009

(54) METHODS FOR GENE MAPPING AND HAPLOTYPING

(76) Inventor: Malcolm James Simons, Bell Post Hil (AU)

Correspondence Address: BIRCH STEWART KOLASCH & BIRCH, PO BOX 747, FALLS CHURCH, VA, 22040-0747, US

(21) Appl. No.: 12/095,907

(22) PCT Filed: Dec. 4, 2006
(86) PCT No.: PCT/AU2006/001836

§ 371 Date: Aug. 19, 2008

Related U.S. Application Data
(60) Provisional application No. 60/741,446, filed on Dec. 2, 2005.

(30) Foreign Application Priority Data
Dec. 2, 2005 (AU) 2005906773

(51) Int. Cl. C12Q 1/68

(52) U.S. Cl. 435/6

(57) ABSTRACT

The present invention is directed to methods for providing a definitive haplotype of a subject. The haplotype information generated by the methods described herein is more accurate than that provided by prior art methods that only give an inferred haplotype. Accordingly, in one aspect the present invention provides a method for determining a definitive haplotype of a subject the method including the steps of providing a substantially isolated haploid element from the subject, and obtaining nucleotide sequence information from the haploid element. Applicants propose that the use of a substantially isolated haploid element eliminates the problem of incorrect or misleading inferences concerning the phase of two or more loci within a haplotype, and allows for revelation of two or more participatory genes within a haplotype, uncomplicated by differences in modes of inheritance. The guarantee of strictly cis-phase associations is provided in the present methods by the use of a substantially isolated haploid element as starting material for sequence analysis.

The present invention relates broadly to the field of genetics. More specifically the invention relates to methods for genomic mapping, and methods for determining the haplotype of a subject.

### BACKGROUND TO THE INVENTION

A genome map describes the order of genes or other markers and the spacing between them on each chromosome. Human genome maps are constructed on several different scales or levels of resolution. At the coarsest resolution are genetic linkage maps, which depict the relative chromosomal locations of DNA markers (genes and other identifiable DNA sequences) by their patterns of inheritance. A genetic linkage map shows the relative locations of specific DNA markers along the chromosome. Any inherited physical or molecular characteristic that differs among individuals and is detectable in the laboratory is a potential genetic marker. Markers can be expressed DNA regions (genes) or DNA segments that have no known coding function but whose inheritance pattern can be followed. DNA sequence differences are especially useful markers because they are plentiful and easy to characterize precisely.

Markers must be polymorphic to be useful in mapping; that is, alternative forms must exist among individuals so that they are detectable among different members in family studies. Polymorphisms are variations in DNA sequence that occur on average once every 300 to 500 bp. Variations within exon sequences can lead to observable changes, such as differences in eye color, blood type, and disease susceptibility. Most variations occur within introns and have little or no effect on the appearance or function of an organism, yet they are detectable at the DNA level and can be used as markers. Examples of these types of markers include (1) restriction fragment length polymorphisms (RFLPs), which reflect sequence variations in DNA sites that can be cleaved by DNA restriction enzymes, and (2) variable number of tandem repeat sequences, which are short repeated sequences that vary in the number of repeated units and, therefore, in length (a characteristic that is easily measured). The human genetic linkage map is constructed by observing how frequently two markers are inherited together.

Two markers located near each other on the same chromosome will tend to be passed together from parent to child. During the normal production of sperm and egg cells, DNA strands occasionally break and rejoin in different places on the same chromosome or on the other copy of the same chromosome (i.e., the homologous chromosome). This process (meiotic recombination) can result in the separation of two markers originally on the same chromosome. The closer the markers are to each other the more tightly linked the less likely a recombination event will fall between and separate them. Recombination frequency thus provides an estimate of the distance between two markers.

The value of the genetic map is that an inherited disease can be located on the map by following the inheritance of a DNA marker present in affected individuals (but absent in unaffected individuals), even though the molecular basis of the disease may not yet be understood nor the responsible gene identified. Genetic maps have been used to find the exact chromosomal location of several important disease genes, including cystic fibrosis, sickle cell disease, Tay-Sachs disease, fragile X syndrome, and myotonic dystrophy.

Current approaches to identifying genes influencing genic functions within a genome have two common characteristics: firstly, non-coding sequence-variant markers are employed to reveal chromosomal regions containing candidate genes; and secondly, genome-wide analyses seek associations between sequence-variant markers and a phenotype of interest. Gene discovery by genome-wide association analysis, also known as linkage disequilibrium mapping, is the subject of U.S. Pat. No. 5,851,762.

While genomic information is useful in linkage studies, information on the haplome is considered more useful for identifying markers of genomic regions of interest in defining disease-associated gene function. The use of haplomic sequences reduces error in linkage studies because there is no need to consider the involvement of second copy of the gene (as provided on the homologous chromosome) with the trait under consideration.

In 2003, a National Institutes of Health-funded international consortium commenced a three year, US$100,000,000 project to create a human genome haplotype map (termed the “HapMap”) in order to facilitate gene discovery by haplotype-based, marker allele-disease gene association in complex diseases. This strategy of linkage disequilibrium allele-association mapping (‘association mapping’) involves unrelated, individual patients, and is the more recent alternative to the traditional approach of family (pedigree) linkage analysis when families are not available.

An assumption upon which HapMap is based is that haplotype inference from genotyping diploid DNA is sufficiently resolving for association mapping to warrant continuation as the strategy for genome-wide gene discovery.

A further assumption is that analysis of SNPs (single nucleotide polymorphisms) of a few hundred individuals from 4 populations (West African Nigerians, Japanese, Chinese, American) will be adequate to identify redundant SNPs, and thereby to identify haplotype-marking single SNPs, or minimum sets of SNPs, sufficient to characterize haplotype blocks in all, including admixed, populations. The HapMap consortium also proposes that only common haplotypes (>5-10%) will be important in common, multigenic diseases and in drug reactivity, and that these common haplotypes will be identifiable in a study of around 200 individuals.

A further assumption is that that the haplome is organized into discrete “blocks” with each block being identifiable with a unique SNP “tag”. It is currently accepted in the field of genetics that use of the minimum essential SNPs revealed by the HapMap project will identify sufficient common haplotypes in any population for detection of excess haplotype sharing in disease-gene searches and drug-affective pharmacogenomics.

Thus, the present state of the art is that the HapMap will be definitive, and capable of providing more than sufficient information for linkage studies. However, closer inspection of the HapMap project suggests that the project will achieve only limited information at best, and may be fundamentally flawed in its application to multi-genic disease discovery. For example, SNP identification will detect only a proportion of extant haplotypes, perhaps not even all commonly occurring haplotypes. Uncommon haplotypes (that may not be detectable in the HapMap) may also contribute to genic functional differences between individuals.

The assumed block structure of the haplome may also lead to errors. Recombinations and other rearrangements can be expected to affect haplotype block structure in admixed populations that may not be revealed by a limited analysis of ‘core’ populations.

The resolving power of inferred haplotypes can be expected to be challenged where two or more genes, having different modes of inheritance (recessive, dominant, co-dominant), differing functions (disease-predisposing, disease-protective), acting at different stages of disease progression, occur within a single chromosomal region. Resolving power will be most challenged when risk is contributed by both chromosomes as in compound heterozygous recessive diseases, and where trans as well as cis co-dominant interactions occur with co-dominant inherited genes such as those of the HLA complex. The significance of these doubts has not been recognized in the art.

A critical test of the utility of haplotyping association mapping is the ability to identify genic regions of interest already identified by pedigree linkage analyses. In an important test case, association mapping failed to identify the 6p21.3 (HLA) region of genetic risk (RR: lower bound 20-upper bound infinity) in nasopharyngeal carcinoma identified by haplotype sharing linkage analysis. This points to another problem in the art: non-coding based strategies have insufficient resolving power to detect even the strongest genetic association with any common human cancer.

Many diseases are known or suspected to be multigenic. Indeed, it is thought that most diseases are multigenic, and that monogenic diseases are the exception. The identification of genes with involvement in multigenic diseases is complicated in the methods of the prior art due to the patterns of inheritance of the genes from the maternal and paternal genotypes. Thus, while the prior art methods of mapping and gene discovery have been useful in identifying genes having simple modes of inheritance and simple involvement in disease, there remains a clear need for more powerful methods to unravel gene involvement in complex diseases.

Accordingly, it is an aspect of the present invention to overcome or at least alleviate a problem of the prior art. In particular, the present invention aims to provide a method for more accurately mapping a gene using haploid information.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention is directed to methods for providing a definitive haplotype of a subject. The haplotype information generated by the methods described herein is more accurate than that provided by prior art methods that only give an inferred haplotype. Accordingly, in one aspect the present invention provides a method for determining a definitive haplotype of a subject the method including the steps of providing a substantially isolated haploid element from the subject, and obtaining nucleotide sequence information from the haploid element. Applicants propose that the use of a substantially isolated haploid element eliminates the problem of incorrect or misleading inferences concerning the phase of two or more loci within a haplotype, and allows for revelation of two or more participatory genes within a haplotype, uncomplicated by differences in modes of inheritance. The guarantee of strictly cis-phase associations is provided in the present methods by the use of a substantially isolated haploid element as starting material for sequence analysis.

In one form of the method the sequence information relates to an allele. In another form of the method the allele is a coding sequence allele.

Applicant recognizes the importance of problems inherent in the use of diploid material in combination with the use of maximum likelihood algorithms in defining a haplotype. It is anticipated that the errors in the methods of the prior art become particularly problematic when investigating traits that have a multi-genic basis.

In one embodiment of the method, the step of substantially isolating a haploid element from the subject involves the use of diploid material as a source for the haploid element. Isolation of a haploid element from the diploid genome or portion thereof may be achieved using any one or combination of methods familiar to the skilled artisan. In one form of the invention, physical methods such as microdissection are used. For example, a chromosome may be microdissected by cutting through the centromere to produce two chromatids.

Separation of the diploid genome or portion thereof may be performed on a diploid cell, such as a somatic cell. The use of naturally haploid material such as sperm cells or ova is to be avoided due to problems with obtaining these sex cells in the clinic. The avoidance of gametes in haplotyping has a further advantage when it is considered that the process of meiosis there are sometimes recombination events such that loci that were formerly linked in cis, become associated in trans. Thus, analysing a haplotype of a gamete will give different (i.e. incorrect) haplotype information to that of a haploid element obtained from a diploid cell.

In another aspect the present invention provides for the use of a haploid element for determining a definitive haplotype of a subject.

In yet a further aspect the present invention provides a method for determining an association between a gene region and a trait, the method including the steps of providing a first set of haploid elements from a plurality of subjects, the individuals being representative of the genetic diversity of a general population, analysing the first set of haploid elements for the presence or absence of an allele, providing a second set of haploid elements from a plurality subjects from the general population, the subjects having the trait, the subjects not derived from a single family, analysing the second set of haploid elements for the presence or absence of the allele, determining the level of allele sharing in the allele between both the first and second sets of haploid elements, wherein excess allele sharing indicates that the allele is associated with the trait. In one form of the method, the allele is a coding sequence allele.

In another aspect the present invention provides a method for identifying a gene involved in a multi-genic disease or trait, the method including use of a method described herein. The provision of a definitive haplotype as described herein removes uncertainties that confound elucidation of the involvement of a single gene in a multi-genic system.

### BRIEF DESCRIPTION OF THE FIGURES

FIG. 1 shows light micrographs of laser pressure catapulting of human chromosome 6. Panels from left to right: metaphase spread, chromosome 6 identification, field post catapult.

FIG. 2 shows light micrographs of a further example of catapulting of human chromosome 6. Left panel, identification; right panel field post catapult.

FIG. 3 shows CFTR exon 10 nested PCR amplicons from chromosome 7 catapults. Lanes 1 to 3, chromosome 7. Lane 4 negative control. Lane 5 positive control.

FIG. 4 shows direct HLA-A exon 2 nested PCR from laser pressure catapulted single chromosome 6s. Lane 1. Single Chromosome 6 Laser Pressure Catapulted into 10 μL 0.1% Triton x-100 Solution. Lane 2. Single Chromosome 6 Laser Pressure Catapulted into 10 μL 0.1% Triton x-100 Solution. Lane 3. Single Chromosome 6 Laser Pressure Catapulted into 10 μL 0.1% Triton x-100 Solution. Lane 4. Single Chromosome 6 Laser Pressure Catapulted into 10 μL 0.1% Triton x-100 Solution. Lane 5. Single Chromosome 6 Laser Pressure Catapulted into 10 μL 0.1% Triton x-100 Solution. Lane 6. Single Chromosome 6 Laser Pressure Catapulted into 10 μL 0.1% Triton x-100 Solution. Lane 7. Single Chromosome 6 Laser Pressure Catapulted into 10 μL 0.1% Triton x-100 Solution. Lane 8. Single Chromosome 6 Laser Pressure Catapulted into 10 μL 0.1% Triton x-100 Solution. Lane 9. Negative Control containing an empty section of PEN membrane captured in 10 μL 0.1% Triton x-100 Solution. Lane 10. Negative Control containing 10 μL 0.1% Triton x-100 Solution only. Lane 11. Positive Control 1 μL 200 pg/μL gDNA in a Total Volume of 10 μL 0.1% Triton x-100 Solution. Lane 12. Molecular Weight Marker VIII. Lane 1, 5, and 6 were submitted for sequencing. Lane 1, 5 and 6 were pure 03 sequences—highlighted by circles.

FIG. 5 shows sequencing of HLA-A amplicons from lanes 1, 5 and 6 of FIG. 4.

FIG. 6 shows DRB1 exon 2 nested PCR amplicons from metaphase laser catapults.

Upper two gels (straddled by the brackets labelled “single metaphases”) show DRB1*01 specific PCR. Lanes of the upper two gels are as follows: Lane 1, markers, Lane 2 (clear band). Single LMPC Metaphase captured into 6 μL 0.1% Triton x-100 Solution. Lane 3. Single LMPC Metaphase captured into 6 μL 0.1% Triton x-100 Solution. Lane 4. Single LMPC Metaphase captured into 6 μL 0.1% Triton x-100 Solution. Lane 5. Negative Control containing 6 μL 0.1% Triton x-100 Solution. Lane 6. Single LMPC Metaphase captured into 6 μL 0.1% Triton x-100 Solution. Lane 7. (clear band) Single LMPC Metaphase captured into 6 μL 0.1% Triton x-100 Solution. Lane 8. (clear band) Single LMPC Metaphase captured into 6 μL 0.1% Triton x-100 Solution. Lane 9. Single LMPC Metaphase captured into 6 μL 0.1% Triton x-100 Solution. Lane 10. (clear band) Single LMPC Metaphase captured into 6 μL 0.1% Triton x-100 Solution. Lane 11. Negative Control containing 6 μL 0.1% Triton x-100 Solution. Lane 12. Single LMPC Metaphase captured into 6 μL 0.1% Triton x-100 Solution. Lane 13. Single LMPC Metaphase captured into 6 μL 0.1% Triton x-100 Solution. Lane 14. Single LMPC Metaphase captured into 6 μL 0.1% Triton x-100 Solution. Lane 15. Single LMPC Metaphase captured into 6 μL 0.1% Triton x-100 Solution. Lane 16. Negative Control containing 6 μL 0.1% Triton x-100 Solution. Lane 17. Single LMPC Metaphase captured into 6 μL 0.1% Triton x-100 Solution. Lane 18 markers. Lane 19 markers. Lane 20. Single LMPC Metaphase captured into 6 μL 0.1% Triton x-100 Solution. Lane 21. Single LMPC Metaphase captured into 6 μL 0.1% Triton x-100 Solution. Lane 22. Single LMPC Metaphase captured into 6 μL 0.1% Triton x-100 Solution. Lane 23. Negative Control containing 6 μL 0.1% Triton x-100 Solution. Lane 24. Single LMPC Metaphase captured into 6 μL 0.1% Triton x-100 Solution. Lane 25 (clear band) Single LMPC Metaphase captured into 6 μL 0.1% Triton x-100 Solution. Lane 26. (clear band) Single LMPC Metaphase captured into 6 μL 0.1% Triton x-100 Solution. Lane 27. Negative Control containing 6 μL 0.1% Triton x-100 Solution. Lane 28. (clear band) Positive Control 1 μL 400 pg/μL gDNA in a Total Volume of 10 μL 0.1% Triton x-100 Solution. Lane 29 markers.

Lower gel shows DRB1*09 specific PCR. Lane 1 markers. Lane 2. Single LMPC Metaphase captured into 6 μL 0.1% Triton x-100 Solution. Lane 3. Negative Control containing 6 μL 0.1% Triton x-100 Solution. Lane 4. Single LMPC Metaphase captured into 6 μL 0.1% Triton x-100 Solution. Lane 5. Negative Control containing 6 μL 0.1% Triton x-100 Solution. Lane 6. (clear band) Single LMPC Metaphase captured into 6 μL 0.1% Triton x-100 Solution. Lane 7. Negative Control containing 6 μL 0.1% Triton x-100 Solution. Lane 8. Single LMPC Metaphase captured into 6 μL 0.1% Triton x-100 Solution. Lane 9. Negative Control containing 6 μL 0.1% Triton x-100 Solution. Lane 10. Single LMPC Metaphase captured into 6 μL 0.1% Triton x-100 Solution. Lane 11. Negative Control containing 6 μL 0.1% Triton x-100 Solution. Lane 12. Single LMPC Metaphase captured into 6 μL 0.1% Triton x-100 Solution. Lane 13. Negative Control containing 6 μL 0.1% Triton x-100 Solution. Lane 14. (clear band) Single LMPC Metaphase captured into 6 μL 0.1% Triton x-100 Solution. Lane 15. Negative Control containing 6 μL 0.1% Triton x-100 Solution. Lane 16. (clear band) Single LMPC Metaphase captured into 6 μL 0.1% Triton x-100 Solution. Lane 17. Negative Control containing 6 μL 0.1% Triton x-100 Solution. Lane 18. Single LMPC Metaphase captured into 6 μL 0.1% Triton x-100 Solution. Lane 19. Negative Control containing 6 μL 0.1% Triton x-100 Solution. Lane 20. Single LMPC Metaphase captured into 6 μL 0.1% Triton x-100 Solution. Lane 21. Negative Control containing 6 μL 0.1% Triton x-100 Solution. Lane 22. (clear band) Positive Control 1 μL 1 ng/μL Pure gDNA in a Total Volume of 10 μL 0.1% Triton x-100 Solution. Lane 23 markers.

FIG. 7 shows dot blot characterisation of DRB1 amplicons derived from chromopults.

### DETAILED DESCRIPTION OF THE INVENTION

In one aspect the present invention provides a method for determining a definitive haplotype of a subject, the method including the steps of providing a substantially isolated haploid element from the subject, and obtaining nucleotide sequence information from the haploid element. Applicants propose that the use of a substantially isolated haploid element eliminates the problem of incorrect or misleading inferences concerning the phase of two or more loci within a haplotype, and allows for revelation of two or more participatory genes within a haplotype, uncomplicated by differences in modes of inheritance.

The present invention is predicated on an improvement over the methods for haplotyping in the prior art, and definitive haplotyping methods described herein. In order to better understand the distinction it is instructive to consider the concept of the haplotype as it is presently understood in the art, and methods for determining the haplotype of a subject.

The term “haplotype” is a contraction of the phrase “haploid genotype”, and is presently accepted to mean a set of nucleotide sequence polymorphisms or alleles present on a single maternal or paternal chromosome, usually inherited as a unit. In the case of a diploid organism such as a human, the haplotype will contain one member of the pair of alleles for a locus.

In the methods of the prior art, determination of a haplotype begins with the use of diploid material that is convenient to obtain clinically. Applicant proposes that the accepted forms of haplotyping using diploid material are inadequate. An alternative is to use naturally haploid material (from sperm or ovum for example) however this is generally inconvenient, and for females is overly invasive. Furthermore, sequence information from gametes can be confounded by crossing over events during meiosis such that cis-phase associations between SNPs cannot be assured to be authentic.

Since approaches utilizing diploid DNA do not allow direct determination of loci linked in cis as haplotypes, occurrence probability of haplotypes is inferred by maximum likelihood and similar algorithm-based estimates. Thus there is always the possibility that two polymorphisms are present in trans phase, and therefore have not been inherited as a single Mendelian unit. The methods of the prior art are therefore more correctly described as providing an “inferred haplotype”. The present methods relate more closely to the strict definition of haplotype by considering the fundamental unit of Mendelian inheritance as a chromosomal segment bounded by sites of parental recombination.

Hitherto, persons skilled in the art have not questioned the effect of errors introduced by the use of diploid starting material. The question of problems in determining trait associations in populations, or in haplotyping a subject have therefore not been recognized. By contrast, Applicant recognizes the importance of problems inherent in the use of diploid material in combination with the use of maximum likelihood algorithms in defining a haplotype. It is anticipated that the errors in the methods of the prior art become particularly problematic when investigating traits that have a multi-genic basis.

Thus, the present invention is directed to methods for providing a definitive haplotype. As used herein, the term “definitive haplotype” is intended to mean that strictly only cis-phase associations are considered in ascribing a haplotype. The methods described herein do not involve any estimation or inference as to whether two polymorphisms are present on the same molecule of DNA. This is different to the “inferred haplotype” described above which is obtained by interrogating diploid material for sequence information (as performed in the HapMap project).

Quite apart from the problems of inference, the use of diploid cells introduces further complications arising from short allele dominance (“allele dropout”) where the larger allele fails to specifically amplify. Thus, the presence of the larger allele is completely ignored, and erroneous haplotype information results.

The guarantee of strictly cis-phase associations is provided in the present methods by the use of a substantially isolated haploid element as starting material for sequence analysis. As used herein, the term “haploid element” is intended to include any nucleic acid molecule (DNA, RNA or derivative thereof) that contains exclusively maternally-derived or paternally-derived nucleotide sequences. The haploid element may be a nucleic acid molecule of any length, including an entire chromosomal length, a chromatid or a portion of a chromatid. The invention may use one or more haploid element for sequence information interrogation. For a human, the method may use all 46 haploid elements, and analyse all 46 haploid elements separately.

The term “substantially isolated” is intended to mean substantially isolated from contaminant genetic material capable of interfering with the identification of only cis-phase associations on the haploid element under interrogation. Typically, where the haploid element under consideration is paternally derived, the contaminant genetic material is a maternally-derived haploid element; and where the haploid element under consideration is paternally derived, the contaminant genetic material is a maternally-derived haploid element.

In one form of the invention the method includes the step of substantially isolating a haploid element from the subject. The means for achieving substantial isolation of the haploid element may be any suitable method known to the skilled artisan. It should be understood that the means for achieving substantial isolation is not restrictive on the scope of the present invention, but in one form of the method the step of substantially isolating the haploid element is by physical means. It will be understood that the haploid element may be isolated from contaminant genetic material by the physical removal of the haploid element away from the contaminant genetic material. This approach may be used where the haploid element is present in a diploid cell. Removal of the haploid element can be achieved by chromosomal micro-dissection, performed either manually or by non-touch instrument manipulation for example. Alternatively, the contaminant genetic material may be physically removed from the haploid element under interrogation.

In another form of the method the contaminant genetic material is inactivated or ablated such that it no longer performs the function of contaminant genetic material. This may be achieved by destroying a homologous chromosome using a carefully directed laser beam for example.

Another possibility is to selectively amplify the haploid element using PCR such that the number of copies of haploid element DNA is in vast excess over that of the contaminant DNA. The mixture of DNA molecules could then be partially digested with a nuclease such that substantially all contaminant DNA is digested, and a low level of haploid element DNA remains.

The possibility also exists for selectively amplifying the haploid element by long PCR using primers incorporating a tag, and separating out the copies using the tag.

The haploid element may be isolated from the genome of a subject or portion thereof. As used herein the term “genome” means the total genetic material of a subject and includes a complete set of DNA sequences of the subject. Reference to a “portion thereof” of a genome means a portion of nucleic acid, such as deoxyribose nucleic acid (DNA), from the genome of a subject.

The genome or portion thereof can be obtained from any cell or biological sample containing nucleic acid. Where the genome is obtained from a diploid cell, the cell may be induced into metaphase by the addition of inducing agents well known to the person skilled in the art such as colcemid. Discrete chromosomes appear at metaphase and are able to be dissected into constituent haplomic elements as described below.

In one embodiment of the method, the step of substantially isolating a haploid element from the subject involves the use of diploid material as a source for the haploid element. Isolation of a haploid element from the diploid genome or portion thereof may be achieved using any one or combination of methods familiar to the skilled artisan. The present invention also includes any other methods that may be developed in the future. In one form of the invention, physical methods such as microdissection are used. A chromosome may be microdissected by cutting through the centromere to produce two chromatids each being a haploid element. Alternatively, cuts in the chromosome may be made distal to the centromere to separate the p and q arms, each being a haploid element. Thus, the haploid element may be a chromatid or a section of a chromatid.

The skilled person is familiar with platforms and tools used for micromanipulation. Although technically exacting, microdissection is achievable in the context of a sophisticated laboratory. Equipment requirements consist of a microscope (either upright or inverted) fitted with a micromanipulator and a rotating stage, a pipette puller (to produce microneedles). Vibration isolation for the microscope is recommended. Although a special clean room is not required, microdissected chromosome fragments contain only femptogram quantities of DNA, and contamination with extraneous DNA must be controlled.

A non-contact method may be used. An example of this approach is the use of a laser microbeam. Laser microbeam microdissection may involve use of a pulsed ultraviolet laser of high beam quality interfaced with a microscope. Laser beam microdissection may be performed using, for example, a commercially available P.A.L.M.® Robot Microbeam (P.A.L.M. GmbH Bernried, Germany). The light laser is typically of a wavelength that does not damage or destroy the genome segment, such as 337 nm which is remote from the absorption maximum of nucleic acids such as DNA.

Another useful system for laser microdissection of chromosomes is the Leica Laser Microdissection Microscope. The system uses a DMLA upright microscope including motorized nosepiece, motorized stage, the xyz-control element and all other advantages of the new DMLA microscope. The laser used is a UV laser of 337 nm wavelength. The movement during cutting is done by the optics, while the stage remains stationary. The region of interest can be marked on the monitor and is cut out by PC control. The sample falls down into PCR tubes without extra forces. The result of the cutting can be easily checked by an automated inspection mode.

Isolation of a haploid element may be achieved by, for example, microdissection using laser catapulting of a chromosome segment using, for example a PALM laser. In this case, the non-contact process involves laser ablation around the targeted chromosome element, followed by laser force catapulting of the defined element onto a tube cap, such as a microcentrifuge tube cap, for subsequent analysis of single arm DNA.

The resultant haploid element(s) may be recovered using laser pressure catapulting. Laser pressure catapulting may be achieved by focussing a laser microbeam under, for example a haploid genome segment or segments of interest, and generating a force as a result of the high photon density that develops and causes the required haploid elements to be catapulted from the non-required genome segment. In one form of the method, the sample travels on the top of a photonic wave and is catapulted into a collection tube. Suitable collection tubes will be known to those of skill in the art and include tubes such as a common polymerase chain reaction (PCR) reaction tube or a microcentrifuge tube.

The haploid element may be substantially isolated by preparative flow cytometry. Another method is by the use of radiation hybrids, where the development of diploid material involves human chromosomes as only one of each chromosome pair. Another strategy is the use of “conversion technology”, as developed by GMP Technologies Inc. GMP Conversion Technology® utilizes a process to separate paired chromosomes into single chromosomes. When separated, alleles may be analyzed individually using genetic probes that identify gene sequences. This technology is applicable to a gene, a chromosome, or to the entire human genome.

Separation of the diploid genome or portion thereof is typically performed on an autosomal chromosome of a somatic cell. The term “autosomal chromosome” means any chromosome within a normal somatic or germ cell except the sex chromosomes. For example, in humans chromosomes 1 to 22 are autosomal chromosomes.

As discussed supra the use of naturally haploid material such as sperm cells or ova is to be avoided due to problems with obtaining these sex cells in the clinic. The avoidance of gametes in haplotyping has a further advantage when it is considered that during the process of meiosis there are sometimes recombination events such that loci that were formerly linked in cis, become associated in trans. Thus, analysing a haplotype of a gamete will give different (i.e. incorrect) haplotype information to that of a haploid element obtained from a diploid cell.

Reference to “obtaining sequence information” is intended to include any method known to the skilled artisan for determining the nucleotide sequence of a nucleic acid molecule, including direct sequencing. A nucleic acid sequence may be obtained using well known techniques such as those described in, for example, Molecular Cloning: A Laboratory Manual by Maniatis et al, Cold Spring Harbor Laboratory 1982. Nucleic acid sequencing may also be performed using automated techniques including use of a range of commercially available instruments. As used herein, the term “nucleic acid” encompasses either or both strands of a double stranded nucleic acid molecule and includes any fragment or portion of an intact nucleic acid molecule. Both DNA and RNA are included.

Sequence information can also be obtained by indirect methods such as by the use oligonucleotide probes. Obtaining sequence information typically involves identification of SNPs, and the skilled person will be capable of designing probes capable of detecting SNPs. Typically, a probe will be around 25 nucleotides in length, with the polymorphic site designed to hybridise with the centre of the probe.

Typically, a precursor step to obtaining sequence information is amplification of the nucleic acid using primers flanking the region of interest. DNA can be obtained from virtually any tissue source (other than pure red blood cells). For example, convenient tissue samples include whole blood, semen, saliva, tears, urine, fecal material, sweat, buccal, skin and hair.

The DNA may be prepared for analysis by any suitable method known to the skilled artisan, including by PCR using appropriate primers. Where it is desired to analyze the entire genome, the method of whole genome amplification (WGA) may be used. For example, anaphase cells may be transacted enabling separation of the two haplomes, after which WGA could be implemented. Commercial kits are readily available for this method including the GenoPlex® Complete WGA kit manufactured by Sigma-Aldrich Corp (St Louis, Mo., USA). This kit is based upon random fragmentation of the genome into a series templates. The resulting shorter DNA strands generate a library of DNA fragments with defined 3 primed and 5 primed termini. The library is replicated using a linear, isothermal amplification in the initial stages, followed by a limited round of geometric (PCR) amplifications.

Numerous strategies are available for PCR amplification of DNA. These include the method described by Klein et al (PNAS 96(8):4494-4499, Apr. 13, 1999) which provides (1) complete, unbiased, whole genome amplification, and (2) the prospect of being able to amplify tens to hundreds of loci within the isolated haploid element.

mRNA samples are also often subject to amplification. In this case amplification is typically preceded by reverse transcription. Amplification of all expressed mRNA can be performed as described in WO 96/14839 and WO 97/01603. Amplification of an RNA sample from a diploid sample can generate two species of target molecule if the subject from whom the sample was obtained is heterozygous at a polymorphic site occurring within expressed mRNA.

A convenient method of identifying nucleotide polymorphisms is by use of microarray technology. An exemplary embodiment of this form of the invention is found in the GeneChip® technology marketed by Affymetrix®. This technology relies on a photolithographic process by coating a 5″×5″ quartz wafer with a light-sensitive chemical compound that prevents coupling between the wafer and the first nucleotide of the DNA probe being created. Lithographic masks are used to either block or transmit light onto specific locations of the wafer surface. The surface is then flooded with a solution containing either adenine, thymine, cytosine, or guanine, and coupling occurs only in those regions on the glass that have been deprotected through illumination. The coupled nucleotide also bears a light-sensitive protecting group, so the cycle can be repeated. Other methods of immobilizing probes are provided by a number of companies including Oxford Gene Technology (Oxford, U.K.), Agilent Technologies (Palo Alto, Calif., U.S.A.) and Nimblegen Systems Inc (Madison, Wis., U.S.A).

It is anticipated that probes designed to provide in formation for both haplotypes of a subject may be incorporated onto a single microarray chip. The probes responsible for providing haplotype information derived paternally may be labelled with one type of fluorescent tag, while haplotype information derived maternally could be labelled with a different tag.

In one form of the invention the two or more polymorphisms are part of a coding sequence allele. The present methods may be used to show an association between a gene and a trait by the demonstration of excess allele sharing. In the context of the present invention the term “allele” is used to refer to a genetic variation associated with a coding region and includes an alternative form of a given gene. As used herein the term “allele sharing” refers to the concept of an allele of a gene being present (or shared) in a group of individuals. The presence and extent of allele sharing can be found using any one of a number of statistical software packages available to skilled person. The use of coding sequence alleles is distinct from the HapMap use of non-coding sequence variants (e.g. SNPs and STRS) for indirect indication of coding haplotypes. This indirect approach by HapMap has inherent uncertainty of SNP representativeness of population haplotype diversity, haplotype block length, linkage disequilibrium and phase. While the invention is not limited to analysis of any particular allele, exemplary alleles are those related to CFTR, DRB1 and HLA-A.

In another aspect the present invention provides for the use of a haploid element for determining a definitive haplotype of a subject.

In yet a further aspect the present invention provides a method for determining an association between a gene region and a trait, the method including the steps of providing a first set of haploid elements from a plurality of subjects, the individuals being representative of the genetic diversity of a general population, analysing the first set of haploid elements for the presence or absence of an allele, providing a second set of haploid elements from a plurality subjects from the general population, the subjects having the trait, the subjects not derived from a single family, analysing the second set of haploid elements for the presence or absence of the allele, determining the level of allele sharing in the allele between both the first and second sets of haploid elements, wherein excess allele sharing indicates that the allele is associated with the trait. In one form of the method the allele is a coding sequence allele.

In another aspect the present invention provides a method for identifying a gene involved in a multigenic disease or trait, the method including use of a method described herein. Given the present methods for haplotyping based on inference it is very difficult if not impossible to fully investigate the role of any given gene in a multi-genic disease or trait. The provision of a definitive haplotype as described herein removes uncertainties that confound elucidation of the involvement of a single gene in a multi-genic system.

It will be apparent to the skilled artisan that the present invention will have many uses. In one embodiment, the present invention provides use of the methods described herein for identifying a gene involved in a monogenic disease or phenotype. A further embodiment provides use of the methods described herein for identifying a gene involved in a multigenic disease or phenotype. The provision of a definitive haplotype will also allow for closer matching of donor and recipients in tissue transplantation.

The present invention can be applied to any subject. The subject may be human, equine, bovine, caprine, ovine, canine, feline, or porcine. The skilled person will understand that organism such as plants will also be suitable. Some organisms are polyploid (e.g. some plants and shellfish) and the present invention is expected to have even greater advantages given the confounding issues involved whereby three or more homologous chromosomes are present per cell.

In a further aspect the present invention provides use of the methods described herein in identifying genes involved in a drug response. Pharmacogenomics is the study of how an individual's genetic inheritance affects the body's response to drugs. The basis of pharmacogenomics is that drugs may be tailor-made for individuals being adapted to their own genetic makeup. Environment, diet, age, lifestyle, and state of health can all influence a person's response to medicines, but understanding an individual's genetic makeup is considered to be the key to creating personalized drugs with greater efficacy and safety.

Using the present invention it may be possible to create drugs based on the proteins, enzymes, and RNA molecules associated with genes and diseases. This will facilitate drug discovery and allow researchers to produce a therapy more targeted to specific diseases. This accuracy not only will maximize therapeutic effects but also decrease damage to nearby healthy cells.

Instead of the standard trial-and-error method of matching patients with the right drugs, clinicians will be able to analyze a patient's genetic profile and prescribe the best available drug therapy from the beginning of therapy. Not only will this take the guesswork out of identifying the correct drug for the patient, it will speed recovery time and increase safety as the likelihood of adverse reactions may be eliminated. Pharmacogenomics has the potential to dramatically reduce the estimated 100,000 deaths and 2 million hospitalizations that occur each year in the United States as the result of adverse drug response.

One result of implementing the present invention may be more accurate methods of determining appropriate drug dosages. Current methods of basing dosages on weight and age will be replaced with dosages based on patient genetics. An accurate genetic profile could, for example, provide an indication of how well the body metabolises the drug and therefore the time taken to metabolize it. This will maximize the therapy's value and decrease the likelihood of overdose.

Another result of implementing the present invention may be improved methods of screening for disease. Knowing the genetic profile of an individual could identify one or more disease susceptibilities. Knowledge of the susceptibility could allow a person to make adequate lifestyle and environmental changes at an early age so as to avoid or lessen the severity of a genetic disease. Likewise, advance knowledge of a particular disease susceptibility will allow careful monitoring of the individual, and treatments can be introduced at the most appropriate stage to optimize therapy.

A further result of implementing the present invention may be improved vaccines. Vaccines made of genetic material, either DNA or RNA, promise the benefits of existing vaccines without all the risks. Genetic vaccines will activate the immune system but will be unable to cause infections. They will be inexpensive, stable, easy to store, and capable of being engineered to carry several strains of a pathogen simultaneously.

The present invention may also be used to improve the drug discovery and approval process. Pharmaceutical companies will be able to discover potential therapies more easily using genome targets. Previously failed drug candidates may be revived as they are matched with the niche population they serve. The drug approval process should be facilitated as trials are targeted for specific genetic population groups providing greater degrees of success. The cost and risk of clinical trials will be reduced by targeting only those persons capable of responding to a drug.

Thus, the provision of accurate genetic information by the present invention may lead to decreases in the number of adverse drug reactions, the number of failed drug trials, the time it takes to achieve regulatory approval for a drug, the length of time a patient is on medication, the number of medications a patient must take to identify an effective therapy, the effects of a disease on the body (through early detection), and an increase in the range of possible drug targets. These advantages may lead to a net decrease in the cost of health care.

As discussed supra, the methods of the present invention may also be useful in identifying gene or genes that render an individual susceptible to a disease. This is especially the case for multi-genic diseases such as diabetes. For example, a major diabetes susceptibility gene has discovered that could make people with defective copies three times more likely to develop type 2 diabetes. It is known that SNPs in the gene for calpain-10 (a protease) are associated with type 2 diabetes. The association was demonstrated in Mexican Americans, who are susceptible to the disease. Sequencing DNA samples from this population and performing statistical analysis on the sequences, it was found that these Mexican Americans had insulin resistance and showed reduced levels of calpain-10 gene expression. The present invention will allow more simple detection of gene/disease associations, and will facilitate identification of the genetic basis for other genetically complex disorders such as asthma, schizophrenia and Alzheimer's disease.

In another aspect the present invention provides use of the methods for identifying gene targets (or protein targets) for drugs or gene therapy. Knowledge of the genetic basis of disease obtained by practicing the present invention will provide valid targets for drug design and gene therapy. For example, if a gene or genes are identified as having an association with a disease, the activity of that gene could be inhibited or enhanced to provide a therapeutic outcome. Alternatively, the protein product of that gene could form the basis of a screening assay for entities that can bind and modulate the activity of the protein. Furthermore, rational drug design could be instigated if the three dimensional structure of the protein is able to be generated.

In another aspect the present invention provides use of the methods described herein for identifying individuals with a predisposition to a disease. Once a gene is identified as having an association with a particular disease using the present methods the skilled person will be able to design an assay to screen for the defective form of the gene. Identification of persons at risk of certain diseases will allow preventative measures such as drug therapy and lifestyle changes to be instigated.

In a further aspect the present invention provides use of the methods to identify individuals with a predisposition to a response to an environmental stimulus. An example of this use could be to identify persons with allergies to various substances. In some individuals, the response to allergens (eg in peanuts or bee venom) can lead to a potentially fatal anaphylactic reaction. Identification of especially sensitive individuals would allow the instigation of desensitization procedures.

The present invention will be described in the following non-limiting examples.

### EXAMPLES

### Example 1

### Laser Microdissection and Pressure Catapulting (LMPC) Procedure for Metaphase Chromosome Isolation at 100× Objective Lens Using Palm Robot

Metaphase spreads were prepared on slides suitable for subsequent Laser Capture using a P.A.L.M microscope under a 100× objective lens. Single metaphases, chromosomes spatially separated from their sister chromosome, including single chromosomes were catapulted into the caps of 200 ul UltraFlux Flat Cap PCR tubes containing 6 μl of 0.1% (v/v) Triton-X-100 using standard P.A.L.M microscope protocols. The catapulted material was transferred to the bottom of the tube by centrifugation for analysis.

### Example 2

### Amplification of Isolated Chromosome

DNA isolated by laser microcapture was amplified by PCR or MDA, using standard protocols. Exon specific PCR amplification protocols for CFTR (exon10), HLA-A (exon2), DRB1 (exon2) follow.

### CFTR

CFTR locus exon10 was amplified using a nested PCR strategy from gDNA, metaphases, or single chromosomes. First round amplification, performed in a 30 μl reaction volume with Taq DNA polymerase, employed primers

| | |
|---|---|
| CF-1F | 5′-GACTTCACTTCTAATGATGAT-3′ |
| and | |
| CF-1R | 5′-CTCTTCTAGTTGGCATGC-3′. |

Under cycling conditions of 95° C. for 3 min; 95° C. for 30 sec, 50° C. for 30 sec, 72° C. for 45 sec—25 cycles; 72° C. for 5 min; 4° C. hold.

A second round nested PCR using the first round products as template is performed using the primers

| | |
|---|---|
| CF-2F | 5′-TGGGAGAACTGGAGCCTT-3′ |
| and | |
| Cf-2r | 5′-GCTTTGATGACGCTTCTGTAT-3′. |

2 μl of the first round was transferred to a 30 μl reaction using Taq DNA polymerase under cycling conditions of; 95° C. for 3 min; 95° C. for 30 sec, 55° C. for 30 sec, 72° C. for 30 sec—40 cycles; 72° C. for 5 min; 4° C. hold.

### HLA-A

The HLA-A locus exon2 was amplified using a nested PCR strategy from gDNA, metaphases, or single chromosomes. First round amplification of HLA-A exon2 was performed using the following generic primers:

SQR2
5′-CTCGGACCCGGAGACTGT-3′
and
M13_5AIn1-46
5′-TGTAAAACGACGGCCAGTGAAACSGCCTCTGYGGGGAGAAGCAA-
3′.

Genomic DNA, single metaphases or single chromosomes were used as the initial template. PCR, employing Platinum Taq DNA polymerase in a 25 μl reaction volume, was carried out under the following cycling conditions; 98° C. for 2 min; 98° C. for 5 sec, 60° C. for 120 sec, 72° C. for 120 sec—10 cycles; 98° C. for 5 sec, 65° C. for 30 sec, 72° C. for 60 sec—25 cycles; 72° C. for 10 min; 4° C. hold.

A second round nested amplification of HLA-A locus exon2, employing 2 μl of the first round in a 25 μl reaction, and cycling conditions of 98° C. for 30 sec; 98° C. for 5 sec, 65° C. for 30 sec, 72° C. for 60 sec—10 cycles; 98° C. for 5 sec, 60° C. for 30 sec, 72° C. for 60 sec—25 cycles; 72° C. for 10 min; 4° C. hold; utilised the following primers:

| | |
|---|---|
| AFE2174B | 5′-TTGGGACGAGGAGACAGGGAAAG-3′, |
| AFE2174E | 5′-GGGACCAGGAGACACGGAATG-3′, |
| AFE2174H | 5′-GGGACGAGGAGACACGGAAGG-3′, |
| AFE2174L | 5′-GGACGGGGAGACACGGAATG-3′, |
| AFE2174C | 5′-TTGGGACCAGGAGACACGGAATA-3′, |
| AFE2174D | 5′-GGACGGGGAGACACGGAAAG-3′, |
| AFE2174F | 5′-GGGACCGGAACACACGGAAWG, |
| AFE2174G | 5′-GGGACCTGCAGACACGGAATG-3′ |
| and | |
| SQR2 | 5′-CTCGGACCCGGAGACTGT-3′. |

### HLA-DRB1

The HLA-DRB1 locus exon2 was amplified using a nested PCR strategy from gDNA, metaphases, or single chromosomes. First round amplification of HLA-DRB1*01 and -DRB1*09 exon2 was performed using specific primers. DRB1*01 was amplified using the following primers:

RB1mf
5′-TGTAAAACGACGGCCAGTTCCCAGTGCCCGCTCCCT-3′
and
RB2mr
5′-CAGGAAACAGCTATGACCACACACTCAGATTCTCCGCTT-3′

DRB1*09 was amplified using the following primers:

I1-RB15mf
5′-TGTAAAACGACGGCCAGTCAGTTAAGGTTCCAGTGCCA-3′
and
I2-RB28mr
5′-CAGGAAACAGCTATGACCACACACACACTCAGATTCCCA-3′.

Genomic DNA, single metaphases or single chromosomes were used as the initial template. PCR employed Platinum Taq DNA polymerase in a 25 μl reaction under cycling conditions of 95° C. for 3 min; 95° C. for 30 sec, 50° C. for 120 sec, 72° C. for 90 sec—30 cycles; 72° C. for 10 min; 4° C. hold.

2 μl this primary reaction was included in a 50 μl second round (nested) reaction using Taq DNA polymerase and degenerate primers

| | |
|---|---|
| GH46V1 | 5′-CCGGATCSTTCGTGTCCCCACAGCAYG-3′, |
| AmpB | 5′-CCGCTGCACTGTGAAGCTCT-3′, |
| AmpB1 | 5′-CCGCTGCACCGTGAAGCTCT-3′, |
| AmpB2 | 5′-CCGCTGCACTGTGAATCTCT-3′ |

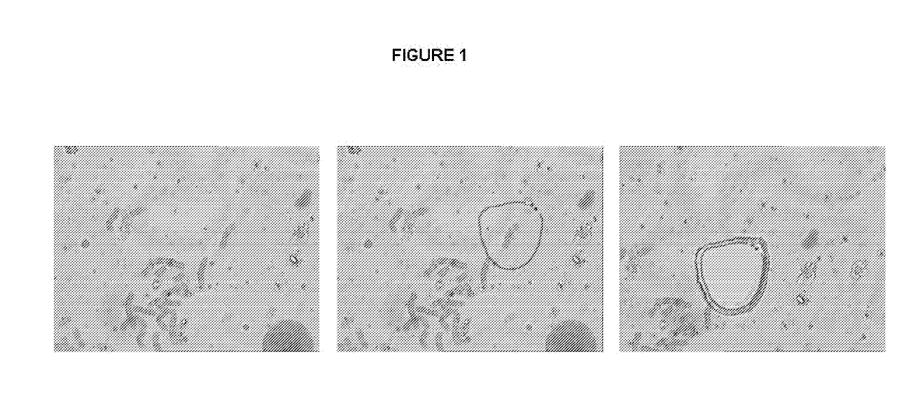

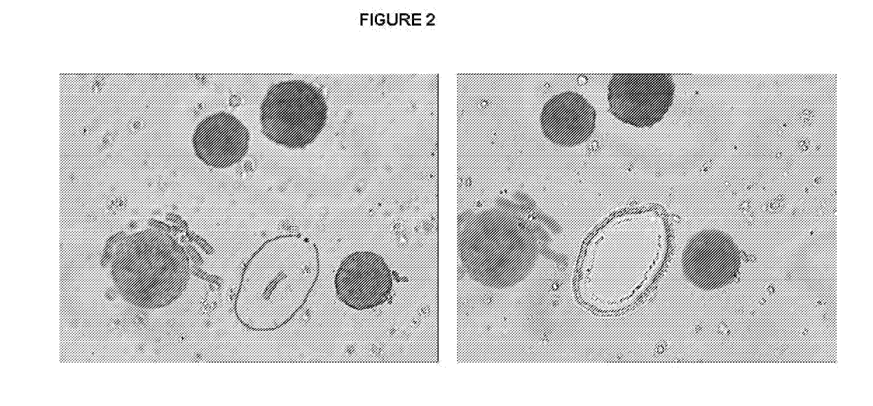

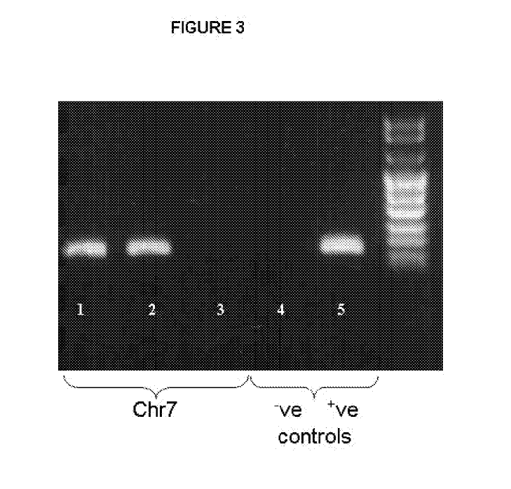

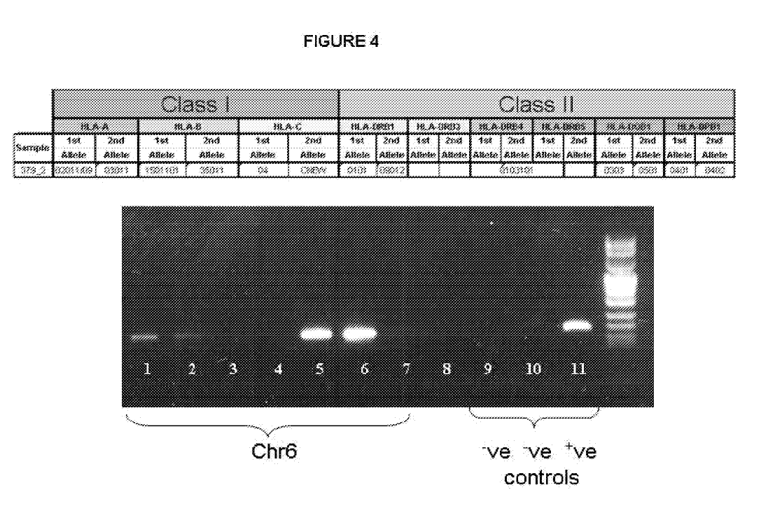

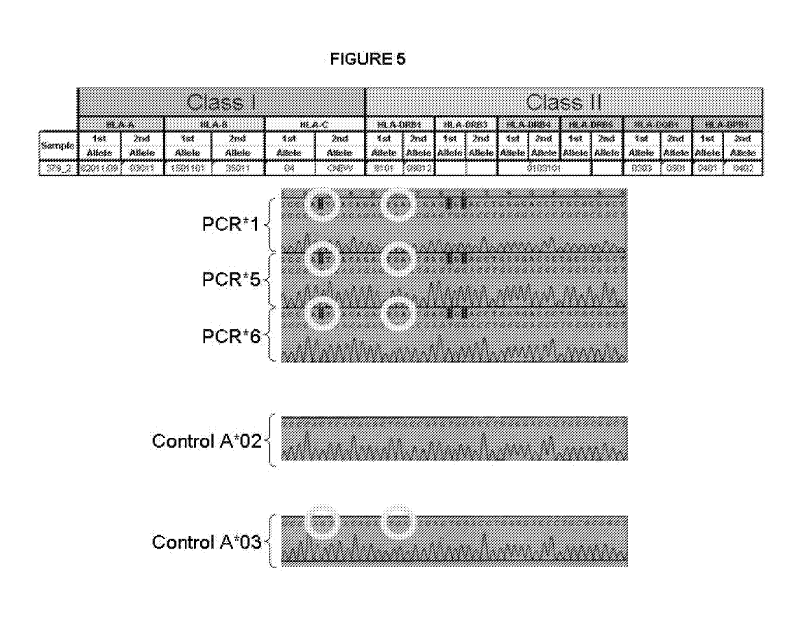

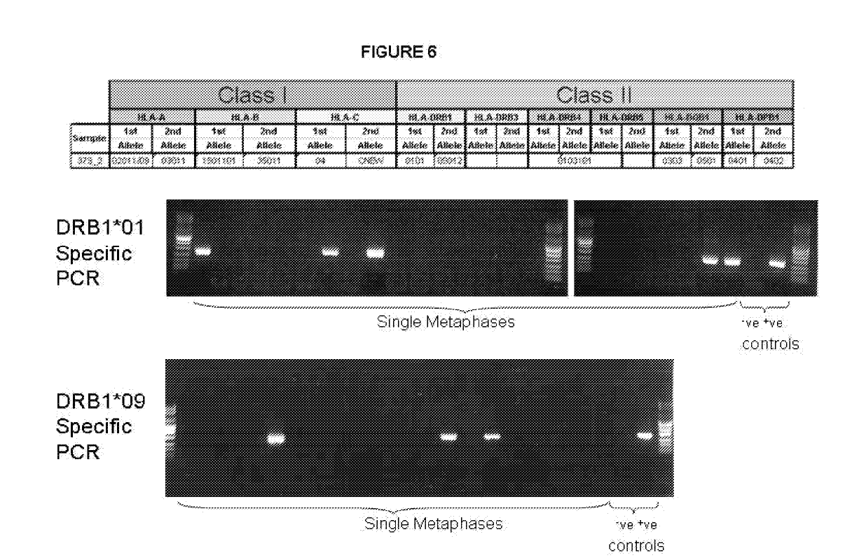

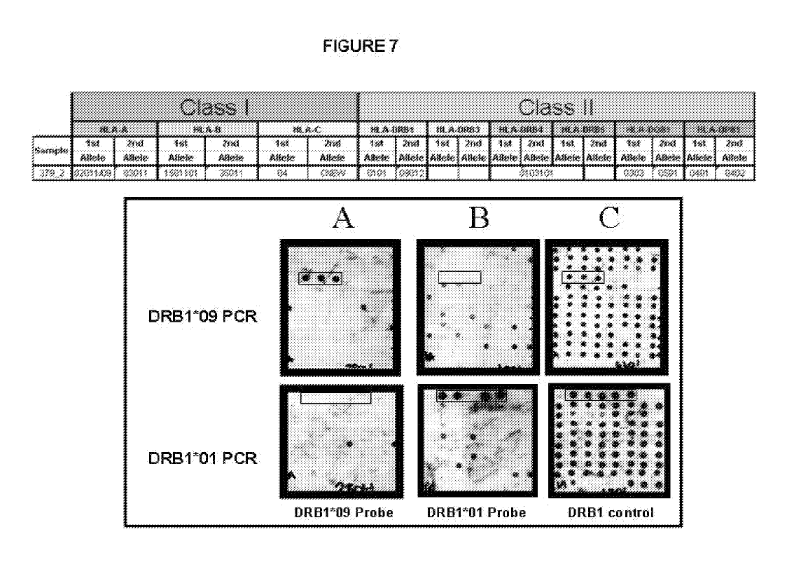

under cycling conditions of; 95° C. for 3 min; 95° C. for 30 sec, 55° C. for 30 sec, 72° C. for 30 sec—30-40 cycles; 72° C. for 5 min; 4° C. hold.
The results of the analyses for CFTR locus 10, HLA-A, HLA-DRB1 are shown in FIGS. 1 to 7. The figures confirm that informative sequence information was obtained from an isolated haploid element using laser micro-dissection and catapulting technique.

Finally, it is to be understood that various other modifications and/or alterations may be made without departing from the spirit of the present invention as outlined herein.

1. A method for determining a definitive haplotype of a subject, the method including the steps of providing a substantially isolated haploid element from the subject, and obtaining nucleotide sequence information from the haploid element.

2. A method according to claim 1 including the step of substantially isolating a haploid element from the subject.

3. A method according to claim 1 wherein the step of substantially isolating the haploid element is by physical means.

4. A method according to claim 3 wherein the physical means is chromosomal micro-dissection.

5. A method according to claim 3 including the step of laser catapulting the haploid element to effect the substantial isolation of the haploid element.

6. A method according to claim 2 wherein the step of substantially isolating a haploid element from the subject involves the use of diploid material as a source for the haploid element.

7. A method according to claim 6 wherein the diploid material is obtained from a somatic cell.

8. A method according to claim 1 wherein the haploid element is a chromatid, or a section of a chromatid.

9. A method according to claim 1 wherein the nucleotide sequence information is the presence or absence of a single nucleotide polymorphism.

10. A method according to claim 1 wherein the nucleotide sequence information provides allelic information.

11. A method according to claim 1 wherein the sequence information is provided by direct sequencing.

12. A method according to claim 1 wherein the sequence information is provided by hybridization with an informative oligonucleotide probe.

13. A method according to claim 12 wherein the oligonucleotide probe detects the presence or absence of a single nucleotide polymorphisim.

14. A method according to claim 13 wherein only c/s-phase single nucleotide polymorphism associations are provided.

15. A method according to claim 10 wherein the allele is a coding region allele.

16. A method according to claim 10 wherein the allele is related to CFTR, DRB1, or HLA-A.

17. Use of a haploid element for determining a definitive haplotype of a subject.

18. A method for determining an association between a gene region and a trait, the method including the steps of providing a first set of haploid elements from a plurality of individuals, said individuals being representative of the genetic diversity of a general population, analysing the first set of haploid elements for the presence or absence of an allele, providing a second set of haploid elements from a plurality of individuals from said general population, said individuals having said trait, said individuals not derived from a single family, analysing the second set of haploid elements for the presence or absence of said allele, determining the level of allele sharing in said allele between for both the first and second sets of haploid elements, wherein excess allele sharing indicates that said allele is associated with the trait.

19. A method according to claim 18 wherein the allele is a coding sequence allele.

20. A method for identifying a gene involved in a multi-genic disease or trait including use of a method according to claim 18.


SEQUENCE LISTING (22 sequences)

SEQ ID NO: 1 - DNA, 21 bp, Artificial Sequence
  primer
gacttcacttctaatgatgat

SEQ ID NO: 2 - DNA, 18 bp, Artificial Sequence
  primer
ctcttctagttggcatgc

SEQ ID NO: 3 - DNA, 18 bp, Artificial Sequence
  primer
tgggagaactggagcctt

SEQ ID NO: 4 - DNA, 21 bp, Artificial Sequence
  primer
gctttgatgacgcttctgtat

SEQ ID NO: 5 - DNA, 18 bp, Artificial Sequence
  primer
ctcggacccggagactgt

SEQ ID NO: 6 - DNA, 44 bp, Artificial Sequence
  primer
tgtaaaacgacggccagtgaaacsgcctctgyggggagaagcaa

SEQ ID NO: 7 - DNA, 23 bp, Artificial Sequence
  primer
ttgggacgaggagacagggaaag

SEQ ID NO: 8 - DNA, 21 bp, Artificial Sequence
  primer
gggaccaggagacacggaatg

SEQ ID NO: 9 - DNA, 21 bp, Artificial Sequence
  primer
gggacgaggagacacggaagg

SEQ ID NO: 10 - DNA, 20 bp, Artificial Sequence
  primer
ggacggggagacacggaatg

SEQ ID NO: 11 - DNA, 23 bp, Artificial Sequence
  primer
ttgggaccaggagacacggaata

SEQ ID NO: 12 - DNA, 20 bp, Artificial Sequence
  primer
ggacggggagacacggaaag

SEQ ID NO: 13 - DNA, 21 bp, Artificial Sequence
  primer
gggaccggaacacacggaawg

SEQ ID NO: 14 - DNA, 21 bp, Artificial Sequence
  primer
gggacctgcagacacggaatg

SEQ ID NO: 15 - DNA, 36 bp, Artificial Sequence
  primer
tgtaaaacgacggccagttcccagtgcccgctccct

SEQ ID NO: 16 - DNA, 39 bp, Artificial Sequence
  primer
caggaaacagctatgaccacacactcagattctccgctt

SEQ ID NO: 17 - DNA, 38 bp, Artificial Sequence
  primer
tgtaaaacgacggccagtcagttaaggttccagtgcca

SEQ ID NO: 18 - DNA, 39 bp, Artificial Sequence
  primer
caggaaacagctatgaccacacacacactcagattccca

SEQ ID NO: 19 - DNA, 27 bp, Artificial Sequence
  primer
ccggatcsttcgtgtccccacagcayg

SEQ ID NO: 20 - DNA, 20 bp, Artificial Sequence
  primer
ccgctgcactgtgaagctct

SEQ ID NO: 21 - DNA, 20 bp, Artificial Sequence
  primer
ccgctgcaccgtgaagctct

SEQ ID NO: 22 - DNA, 20 bp, Artificial Sequence
  primer
ccgctgcactgtgaatctct